(19) United States
(12) Patent Application Publication
(10) Pub. No.: US 2025/0182507 A1
(43) Pub. Date: Jun. 5, 2025

(54) MACHINE LEARNING ARCHITECTURES TO ANALYZE IMAGING DATA

(71) Applicant: HistAl, Inc., Dover, DE (US)

(72) Inventors: Alexey Pchelnikov, Mougins (FR); Alexei Pchelnikov, Batumi (GE); Dmitrii Nechaev, Tbilisi (GE); Valentin Samokhin, Istanbul (TR); Yan Smaliak, Warsaw (PL); Ekaterina Ivanova, Tbilisi (GE)

(21) Appl. No.: 18/962,785

(22) Filed: Nov. 27, 2024

Related U.S. Application Data
(60) Provisional application No. 63/604,599, filed on Nov. 30, 2023.

(51) Int. Cl. G06V 20/69; G06V 10/764; G06V 10/77; G06V 10/774; G06V 10/776; G06V 10/94; G16H 15/00

(52) U.S. Cl. CPC G06V 20/698; G06V 10/764; G06V 10/7715; G06V 10/774; G06V 10/776; G06V 20/695; G16H 15/00; G06V 10/945; G06V 2201/03

(57) ABSTRACT

Implementations described herein are directed to the analysis of biological images to identify subjects in which a biological condition is present. The biological images can be analyzed by implementing one or more machine learning techniques. For example, one or more transformer models can be implemented to analyze biological images to identify subjects in which a biological condition is present. In one or more examples, chat operations can be integrated with the analysis of biological images to access various types of functionalities that can be performed with respect to the analysis of biological images, such as identifying images that are indicative of a biological condition, generating reports based on the image analysis, and navigating workflows related to the analysis of biological images.

### CLAIM OF PRIORITY

This application claims priority to U.S. provisional patent application Ser. No. 63/604,599, filed Nov. 30, 2023, and entitled MACHINE LEARNING ARCHITECTURES TO ANALYZE BIOLOGICAL IMAGING DATA, which is incorporated by reference herein in its entirety.

### BACKGROUND

Tissue samples can be obtained from subjects and analyzed to determine features and characteristics of the tissue. In some cases, the analysis of tissue samples can be used to identify tissue samples that may be indicative of a biological condition. For example, tissue samples can be analyzed to determine whether or not a given disease is present in the tissue. Tissue samples can be prepared for analysis through a staining process that can highlight various features of the tissue. To illustrate, tissue samples can be dyed to identify diseased cells, such as cells that are cancerous. Histology slides can include tissue samples and be examined under a microscope. In many situations, physical histology slides are scanned using a scanning device that produces digital images of the histology slides. In at least some cases, the process of scanning physical histology slides to produce digital images of the slides can be referred to as whole slide imaging.

### BRIEF DESCRIPTION OF THE DRAWINGS

The present disclosure is illustrated by way of example and not limitation in the figures of the accompanying drawings, in which like references indicate similar elements.

FIG. 1 is a diagram illustrating an example architecture to analyze biological imaging data, in accordance with one or more implementations.

FIG. 2 is a diagram illustrating an example cloud computing architecture to analyze biological imaging data, in accordance with one or more implementations.

FIG. 3 is a flow diagram illustrating an example process to analyze biological imaging data, in accordance with one or more implementations.

FIG. 4 is a block diagram illustrating components of a machine, in the form of a computer system, that may read and execute instructions from one or more machine-readable media to perform any one or more methodologies described herein, in accordance with one or more example implementations.

FIG. 5 is a block diagram illustrating a representative software architecture that may be used in conjunction with one or more hardware architectures described herein, in accordance with one or more example implementations.

FIG. 6 illustrates a comparison of a number of classification metrics for an example architecture described herein that analyzes biological imaging data with respect to an existing architecture that analyzes biological imaging data.

FIG. 7 illustrates a comparison of recall/sensitivity for an example architecture described herein that analyzes biological imaging data with respect to an existing architecture that analyzes biological imaging data.

### DETAILED DESCRIPTION

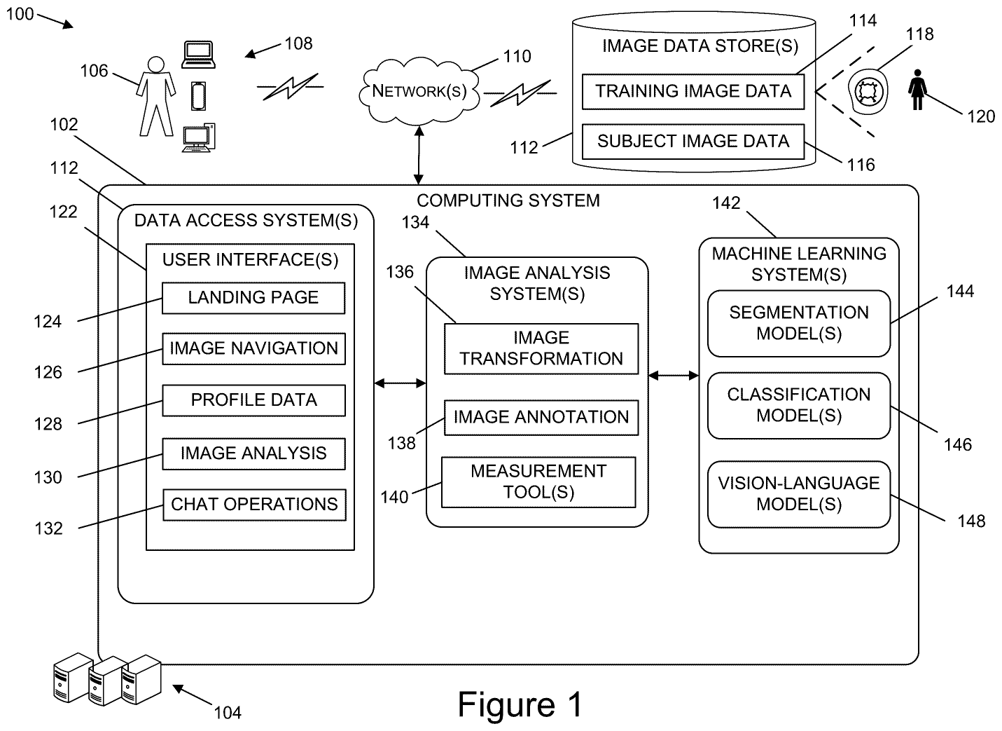

FIG. 1 is a diagram illustrating an example architecture 100 to analyze biological imaging data, in accordance with one or more implementations. The architecture 100 can include a computing system 102. The computing system 102 can analyze biological imaging data. For example, the computing system 102 can analyze whole slide images generated from histology slides. In various examples, the computing system 102 can analyze biological imaging data to determine the presence or absence of one or more biological conditions with respect to a subject. As used herein, a biological condition can refer to an abnormality of function and/or structure in an individual to such a degree as to produce or threaten to produce a detectable feature of the abnormality. A biological condition can be characterized by external and/or internal characteristics, signs, and/or symptoms that indicate a deviation from a biological norm in one or more populations. A biological condition can include at least one of one or more diseases, one or more disorders, one or more injuries, one or more syndromes, one or more disabilities, one or more infections, one or more isolated symptoms, or other atypical variations of biological structure and/or function of individuals. In some instances, described herein, a biological condition can be referred to as a “biological issue.” Additionally, a treatment, as used herein, can refer to a substance, procedure, routine, device, and/or other intervention that can be administered or performed with the intent of alleviating one or more effects of a biological condition in an individual.

In various examples, the architecture 100 can be implemented to couple the analysis of imaging data to generating at least one of text or image outputs using one or more generative machine learning models that is not present in existing systems. The coupling of the analysis of the imaging data with the generative functionality is achieved through a number of training processes and features related to the architectures of the machine learning models implemented in the embodiments described herein. In various examples, the analysis of the imaging data and the output from the one or more generative machine learning models can be directed to providing information related to one or more biological conditions, such as the presence of one or more proteins or one or more genomic regions that can be indicative of one or more types of cancer. The techniques, processes, and architectures described herein improve the functioning of systems that implement the techniques, processes, and architectures by increasing the speed of the training process and reducing the amount of processing and memory resources used during the training of the machine learning architectures and during the implementation of the machine learning architectures to analyze imaging data in relation to a biological condition and provide text and/or image output related to the analysis.

The computing system 102 can be implemented by one or more computing devices 104. The one or more computing devices 104 can include one or more server computing devices, one or more desktop computing devices, one or more laptop computing devices, one or more tablet computing devices, one or more mobile computing devices, or combinations thereof. In certain implementations, at least a portion of the one or more computing devices 104 can be implemented in a distributed computing environment. For example, at least a portion of the one or more computing devices 104 can be implemented in a cloud computing architecture.

In one or more examples, a practitioner 106 can access the computing system 102 using one or more computing devices 108. In at least some examples the practitioner 106 can include a doctor, a nurse, a technician, a practitioner assistant, or another healthcare worker. The one or more computing devices 108 can include one or more mobile computing devices, one or more smart phones, one or more table computing devices, one or more laptop computing devices, one or more desktop computing devices, one or more additional computing devices, or one or more combinations thereof. The one or more computing devices 108 can be in communication with the computing system 102. The computing system 102 can facilitate the communication of various types of information between the one or more computing devices 108 and the computing system 102. In various examples, the one or more practitioner computing devices 108 can execute a mobile device application and/or a browser application that can process and display information obtained from the computing system 102. In one or more illustrative examples, the one or more computing devices 108 can be in communication with the computing system 102 via one or more networks 110. The one or more networks 110 can be representative of any one or combination of multiple different types of wired and wireless networks, such as the Internet, cable networks, cellular networks, satellite networks, wide area wireless communication networks, wireless local area networks, wired local area networks, and public switched telephone networks (PSTN).

The computing system 102 can include one or more data access systems 112. The one or more data access systems 112 can provide access to functionality implemented by the computing system 102 and to data that can be retrieved and/or stored by the computing system 102. In one or more examples, the data access system 112 can implement operations and computational features of an application that is executable to view and analyze data related to biological images. In various examples, the application can include a web-based application that is accessible to the one or more computing devices 108 or an instance of a user device application that is executed by the one or more computing devices 108.

In addition, the computing system 102 can be in electronic communication with one or more image data stores 112 via the one or more networks 110. In one or more examples, the one or more image data stores 112 can store training image data 114 and subject image data 116. The training image data 114 and the subject image data 116 can include images that correspond to tissue samples obtained from one or more subjects, such as example tissue sample 118 obtained from example subject 120. The images corresponding to the samples 118 can be obtained by scanning histology slides that include at least a portion of the samples 118. In at least some examples, the histology slides can be stained in relation to at least one of one or more proteins or one or more genomic regions that can be indicative of one or more biological conditions. To illustrate, the histology slides can be stained with respect to at least one of one or more proteins or one or more genomic regions that can be indicative of one or more types of cancer. In one or more illustrative examples, the histology slides can be stained in relation to at least one of one or more proteins or one or more genomic regions indicative of skin cancer being present with respect to a subject. In one or more additional illustrative examples, the histology slides can be stained in relation to at least one of Ki67 antibodies, HMB45 antibodies, SOX10 transcription factors, Melan-A proteins, S100 proteins, or hematoxylin and eosin.

In one or more examples, the histology slides can be scanned by one or more scanning devices to generate the training image data 114 and the subject image data 116. In one or more illustrative examples, the one or more scanning devices used to scan the histology slides that include at least a portion of the samples 118 to produce the training image data 114 and the subject image data 116 can include at least one of scanning devices manufactured by Leica, Siemens, Hamamatsu, 3DHISTECH, and Philips. Other scanning devices that may be used to generate the training image data 114 and the subject image data 116 can include scanning devices manufactured by Grundium Ocus, Huron, Morphle, MoticEasyScan, and Optrascan. In various examples, the training image data 114 and the subject image data 116 can include images formatted according to one or more format types. To illustrate, the training image data 114 and the subject image data 116 can be formatted according to at least one of a .tif format, a .sys format, a .dcm format, a .vms format, a .vmu format, a .ndpi format, a .scn format, a .mrxs format, a .syslide format, or a .bif format.

In various examples, the training image data 114 can include images that can be used to train and validate one or more computational models implemented by the computing system 102 that analyze biological imaging data and generate output based on the analysis. The subject image data 116 can include images of tissue samples 118 obtained from subjects 120 that are to be analyzed by the computing system 102 with respect to one or more biological conditions being present or absent in a number of subjects 120. In at least some examples, the training image data 114 can include images that can be used to train and validate one or more machine learning models implemented by the computing system 102. In one or more additional examples, the training image data 114 can include at least one of annotations or classifications that correspond to individual images included in the training image data 114. For example, the training image data 114 can include annotations obtained from a healthcare practitioner, such as a radiologist, indicating a first portion of the images included in the training image data 114 that are indicative of a biological condition being present in a subject 120 and a second portion of the images included in the training image data 114 thar are indicative of a biological condition being absent from a subject 120. In one or more examples, the training image data 114 can also include information indicating features of images, sizes of image features, distances between image features, observations based on image features, one or more combinations thereof, and the like.

The one or more data access systems 112 can generate one or more user interfaces 122 that can display biological imaging data and that can include user interface elements that are selectable to cause one or more operations to be performed with respect to the biological imaging data. The one or more user interfaces 122 can include a landing page 124. The landing page 124 can include a user interface that is displayed in response to a user launching an instance of an application that accesses the functionality provided by the computing system 102. The landing page 124 can include one or more user interface elements to obtain information stored by the one or more image data stores 112 and/or to execute computational operations with respect to information stored by the one or more image data stores 112. In one or more examples, the landing page 124 can include one or more user interface elements directed to image navigation 126. In one or more additional examples, functionality related to image navigation 126 can be accessed via one or more additional user interfaces. Image navigation 126 can include accessing one or more images stored by the one or more image data stores 112, such as one or more images included in the subject image data 116. Image navigation 126 can also include search functionality with respect to one or more images stored by the one or more image data stores 112. For example, one or more of the user interfaces 122 directed to image navigation 126 can include one or more user interface elements to search for images and/or groups of images assigned to one or more practitioners 106. Additionally, one or more of the user interfaces 122 directed to image navigation 126 can include one or more user interface elements to search for images having a given image review status. In one or more illustrative examples, the given image review status can include “In Process”, “In Review”, “Completed” or one or more combinations thereof. Further, one or more user interfaces 122 directed to image navigation 126 can include one or more user interface elements corresponding to searching for images stored by the one or more image data stores 112 that correspond to one or more subjects 120.

In one or more additional examples, the landing page 124 can include one or more user interface elements directed to accessing profile data 128 of users of the computing system 102. To illustrate, the landing page 124 or one or more additional user interfaces related to accessing profile data 128 can include one or more user interface elements that are selectable to access at least one of settings for one or more users of the computing system 102, activity of one or more users of the computing system 102, or personal information of one or more users of the computing system 102.

The one or more user interfaces 122 can also include user interface elements that correspond to image analysis 130. In one or more examples, user interface elements related to image analysis 130 can be selectable to initiate the implementation of one or more machine learning algorithms to determine that a biological condition is present with respect to a sample 118. In one or more additional examples, user interface elements related to image analysis 130 can be selectable to determine information about one or more features of an image. For example, the one or more user interfaces 122 related to image analysis 130 can include one or more user interface elements selectable to measure one or more features included in an image. In still other examples, the one or more user interfaces 122 related to image analysis 130 can include one or more user interface elements that are selectable to generate annotations related to one or more images being analyzed.

In at least some examples, chat operations 132 can be integrated with the one or more user interfaces 122. In various examples, the chat operations 132 can be accessed to provide at least one of text input, video input, or audio input that corresponds to one or more commands, one or more phrases, or one or more questions related to images stored by the one or more image data stores 112. In one or more examples, the chat operations 132 can be implemented to perform image navigation 126 and/or to access profile data 128. In one or more additional examples, the chat operations 132 can be implemented to perform one or more image analysis operations. In one or more illustrative examples, the chat operations 132 can be implemented to request that a biological image is analyzed with respect to one or more biological conditions. In one or more additional illustrative examples, the chat operations 132 can be implemented to request that a report is generated that includes results of an analysis of a biological image. In one or more further illustrative examples, the chat operations 132 can be implemented to indicate a status of an image and/or to indicate that a workflow related to an image can proceed to one or more additional steps of the workflow. In still other illustrative examples, the chat operations 132 can be implemented to access materials and/or information to aid a user of the computing system 102 to perform analyses of biological images.

The computing system 102 can also include one or more image analysis systems 134. The one or more image analysis systems 134 can perform operations related to the viewing and modification of one or more images stored by the one or more image data stores 112. For example, the one or more image analysis systems 134 can perform image transformation 136. Image transformation 136 can include modifying an amount of magnification of one or more biological images displayed in the one or more user interfaces 122. To illustrate, image transformation 136 can include increasing an amount of magnification with respect to one or more biological images or decreasing an amount of magnification with respect to one or more biological images. Additionally, image transformation 136 can include panning operations to focus on a portion of a biological image displayed in the one or more user interfaces 122. Further, image transformation 136 can include rotating at least one biological image displayed in the one or more user interfaces 122.

The one or more image analysis systems 134 can also perform image annotation 138. The image annotations 138 can include one or more masks and/or one or more outlines of features shown in one or more biological images displayed in the one or more user interfaces 122. In at least some examples, the image annotations 138 can highlight one or more areas of a biological image displayed in the one or more user interfaces 122 based on staining of a histology slide on which the biological image was based. In various examples, the image annotations 138 can indicate one or more areas of interest for a biological image displayed in the one or more user interfaces 122. In one or more illustrative examples, the image annotations 138 can indicate one or more areas of a biological image that can be indicative of one or more biological conditions. To illustrate, the image annotations 138 can indicate one or more areas of a biological image that are indicative of cancer being present in a subject. In addition, the image annotations 138 can include markings made using one or more markup tools with respect to a biological image displayed in the one or more user interfaces 122. In one or more illustrative examples, the image annotations 138 can include notes, symbols, characters, shapes, one or more combinations thereof, and so forth made by implementing one or more markup tools supported by the one or more image analysis systems 134.

In various examples, the one or more image analysis systems 134 can include one or more measurement tools 140. The one or more measurement tools 140 can include one or more graphical tools and one or more computational tools to measure distances between objects included in a biological image displayed in the one or more user interfaces 122. For example, the one or more measurement tools 140 can be implemented to measure distances between one or more areas of interest of a biological image displayed in the one or more user interfaces 122. In one or more additional examples, the one or more measurement tools 140 can be implemented to determine dimensions or other measurements with respect to areas of interest included in biological images, such as at least one of length, width, diameter, or area of areas of interest.

In at least some examples, at least a portion of the operations performed by the one or more image analysis systems 134 can be integrated with the chat operations 132. In one or more examples, one or more image transformations 136 can be performed with respect to a biological image in response to at least one of one or more commands, one or more phrases, or one or more words received in relation to the chat operations 132. In addition, one or more image annotations 138 can be performed with respect to a biological image in response to at least one of one or more additional commands, one or more additional phrases, or one or more additional words received in relation to the chat operations 132. Further, one or more measurement tools 140 can be implemented with respect to a biological image in response to at least one of one or more further commands, one or more further phrases, or one or more further words received in relation to the chat operations 132.

Additionally, the computing system 102 can include one or more machine learning systems 142. The one or more machine learning systems 142 can implement one or more machine learning algorithms to analyze features of biological images and generate output that can be indicative of a biological condition being present with respect to a subject. In at least some examples, the one or more machine learning systems 142 can be implemented, at least in part, by one or more application specific integrated circuits (ASICs). The one or more machine learning systems 142 can include one or more segmentation models 144. The one or more segmentation models 144 can implement one or more machine learning algorithms to determine portions of a biological image that correspond to tissue. The one or more segmentation models 144 can also implement one or more machine learning algorithms to segment the biological image into regions that include the areas of the biological image including tissue. In this way, in at least some examples, non-tissue regions can be omitted after the segmentation process. Further, the one or more segmentation models 144 can generate output that indicates on a per-pixel basis a probability of an individual pixel indicating the presence of a biological condition in a subject. In various examples, the one or more segmentation models 144 can generate a feature map that aggregates the probabilities for individual pixels of the biological image that the individual pixels indicate the presence of the biological condition in the subject. In one or more illustrative examples, the one or more segmentation models 144 can include one or more transformer architectures. In one or more additional illustrative examples, the one or more segmentation models 144 can include a UperNet architecture and an EVA-02 architecture implemented as a feature extractor.

The one or more segmentation models 144 can be training using the training image data 114. In one or more examples, the training process for the one or more segmentation models 144 can include dividing individual biological images included in the training image data 114 into a matrix of patches at a level of magnification that is increased with respect to an initial level of magnification of the individual biological images included in the training image data 114. In at least some examples, the level of resolution of the matrix of patches can be less than an initial level of resolution of the individual biological images included in the training image data 114. In one or more illustrative examples, the one or more segmentation models 144 can be trained using a first number of patches extracted from biological images included in the training image data 114 that indicate the presence of a biological condition and a second number of patches extracted from biological images included in the training image data 114 that are indicative of the absence of the biological condition. In one or more additional illustrative examples, the first number of patches and the second number of patches can be at least approximately equal. The use of approximately equal numbers of patches that indicate the presence of the biological condition and numbers of patches that indicate the absence of the biological condition can minimize the possibility of class disbalance because the majority of patches derived from the image training data 114 do not indicate the presence of a biological condition, such as the presence of a tumor.

In one or more examples, the training of the one or more segmentation models 144 can include determining values of one or more loss functions to determine when to stop the training process for the one or more segmentation models 144. In various examples, the one or more loss functions used in the training of the one or more segmentation models 144 can include focal loss and/or dice loss. In one or more additional examples, optimization of the loss functions can be performed using an AdamW optimizer. During training of the one or more segmentation models 144 values for a number of training metrics can be determined for each epoch of the training process and the parameters of the one or more segmentation models 144 during the training process that result in the metrics having optimal values are used as the parameters for the trained versions of the one or more segmentation models 144. In one or more illustrative examples, the metrics generated during the training process for the one or more segmentation models 144 can include at least one of intersection over union (IoU) values, accuracy values, recall values, or F1.

The one or more machine learning systems 142 can also include one or more classification models 146. In at least some examples, an output of the one or more classification models 146 can include an indication of a biological condition being present in a subject. The indication of a biological condition being present in a subject can include a binary value, such as yes or no. In one or more additional examples, the indication of a biological condition being present in a subject can include a probability of a biological condition being present. In one or more further examples, the indication of a biological condition being present in a subject can include a value on a scale, such as 0 to 100, where higher values on the scale indicate a greater probability of a biological condition being present in a subject. In various examples, a feature map generated by the one or more segmentation models 144 can be provided as input for the one or more classification models 146.

In at least some examples, the one or more biological conditions being detected by the one or more machine learning systems 142 can include one or more types of melanoma. For example, the one or more biological conditions being detected by the one or more machine learning systems 142 can include cutaneous melanoma. In various examples, the one or more machine learning systems 142 can classify a subject as having melanoma or as having one or more nevi. In at least some examples, the melanoma can be present on the skin of a subject. The melanoma can also be present with respect to other organs and/or body parts of subjects, such as the eyes, ears, gastrointestinal tract, leptomeninges, oral mucous membranes, genital mucous membranes, or one or more combinations thereof. In one or more additional examples, the one or more machine learning systems 142 can determine output indicating the presence or absence of cancerous melanocytic lesions or non-cancerous melanocytic lesions. The one or more machine learning systems 142 can also generate output indicating the presence or absence of other types of cancer, such as one or more colorectal cancers or one or more lymphatic cancers. The one or more colorectal cancers can include at least one of adenocarcinoma, melanomas, carcinoids, lymphoma, gastrointestinal stromal tumors, or leiomyosarcomas.

In one or more examples, the one or more classification models 146 can include one or more transformer models. In various examples, the one or more classification models 146 can be implemented using a transformer architecture with a trainable cls token and without positional encoding. In at least some examples, input data provided to the one or more classification models 146 can have varying lengths. In one or more illustrative examples, the one or more classification models 146 can be provided with an input sequence with no elements of the input sequence being masked.

In various examples, the training of the one or more classification models 146 can include determining values of one or more loss functions. In at least some examples, the one or more loss functions can include a cross-entropy loss function. The training of the one or more classification models 146 can also include implementing label smoothing to minimize or prevent model overfitting. In one or more examples, an AdamW optimizer is used to optimize the training of the one or more classification models 146. In one or more illustrative examples, a training sequence corresponding to one or more biological images included in the image training data 114 can be shuffled with some tokens being masked. One or more metrics can be determined during training of the one or more classification models 146 to determine when to stop the training process and to identify the final weights of the trained versions of the one or more classification models 146. In at least some examples, the one or more metrics can include at least one of accuracy, precision, recall, specificity, and Jaccard Index. The one or more metrics can be calculated for each epoch of the training process and the training process can be completed in response to determining a greatest Jaccard Index for a set of parameters of the one or more classification models 146.

In one or more examples, the training of the one or more classification models 146 can include using parameter efficient fine tuning (PEFT). The use of PEFT can alleviate overfitting during training of the one or more classification models 146. In addition, Low-Rank Adaptation (LoRA) techniques can also be used in the training of the one or more classification models 146 for fine-tuning of the one or more classification models 146. In at least some examples,

LoRA techniques can be applied to attention matrices of individual layers of a transformer model. The LoRA techniques can be applied with a dropout rate of 0.05, 0.1, 0.2, 0.3, 0.4, or 0.5. The patch embedding layer and the final classifier block can be fine tuned independently. The fine-tuning processes can be performed in order to cause the one or more classification models 146 to provide accurate and efficient classification in histopathological applications. In various examples, a focal loss function can be implemented during the training of the one or more classification models 146. The training dataset can be balanced using weighted sampling strategies. Additionally, images included in the training data can be augmented. The augmentation techniques applied with respect to training images can include affine transformations and/or elastic transformations. At least one of brightness, blur, jitter, or sharpen augmentations can also be applied to the training data. In various examples, the augmentations to images included in the training data can be used to simulate images corresponding to a number of quality and/or distortion characteristics.

Additionally, the one or more machine learning systems 142 can include one or more vision-language models 148. The one or more vision-language models 148 can be implemented to generate words, characters, phrases, sentences, one or more combinations thereof and the like with respect to features of biological images. In one or more examples, the one or more vision-language models 148 can be implemented to generate reports that correspond to the analysis of biological images. The one or more vision-language models 148 can be trained using training image data 114 that includes whole slide images derived from tissue samples 118 and that includes information about whole slide images in conjunction with histopathological conclusions derived from the information about the whole slide images. In various examples, the histopathological conclusions about the whole slide images can be obtained from one or more healthcare practitioners. In at least some examples, the one or more vision-language models 148 can be trained using whole slide images included in the training image data 114 for which one or more healthcare practitioners have confirmed correspond to subjects in which a biological condition is present. In one or more illustrative examples, the one or more vision-language models 148 can be trained using first whole slide images included in the training image data 114 that are marked as indicating melanoma and second whole slide images that are marked as indicating nevi including dyplastic nevus, blue nevus, and/or Sptiz nevus.

In at least some examples, during the training process of the one or more vision-language models 148, features extracted from the image space using a feature extraction model can be aligned with text features using a linear projection layer. In one or more examples, the training process used to align the image features and language features can implement a cross-entropy loss function. The linear projection layer can further be trained by adding low rank adapters to the weights of a large vision language model. In this portion of the training process, cross entropy loss can also be used. Based on a correlation between the language portion of the one or more vision-language models 148 during training with respect to image features included in the image training data 114, an indication of accuracy can be determined. The indication of accuracy of the language-based output of the one or more vision-language models 148 can be used to implement proximal policy optimization techniques to further train the weights of the one or more vision-language models 148.

In various examples, the one or more vision-language models 148 can be trained to extract information from the descriptions of the whole slide images included in the training image data 114 that can be used to generate reports for images included in the subject image data 116 that have features related to the training image data 114. In at least some examples, the one or more vision-language models 148, can implement chain-of-thought techniques to extract information from the descriptions of whole slide images included in the training image data 114 to generate reports for images included in the subject image data 116. In one or more examples, the one or more vision-language models 148 can be implemented in response to input obtained via the chat operations 132 related to at least one of commands, phrases, or questions corresponding to generating a report for an image included in the subject image data 116 that is being viewed via the one or more user interfaces 122.

In one or more illustrative examples, the one or more vision-language models 148 can include a feature extraction model and a large vision language model. After the training process, the feature extraction model can generate a sequence of feature vectors that correspond to an image included in the subject image data 116 and provide the sequence of feature vectors to the large vision language model. The large vision language model can then analyze the feature vectors with respect to a corpus of text that corresponds to at least one of observations or conclusions that correspond to histopathology of whole slide images. Based on the feature vectors, the large vision language model can identify at least one of observations or conclusions related to the image being analyzed and generate a report using the identified observations and/or conclusions. In various illustrative examples, the feature extraction model can include an EVA-02 transformer model and the large language vision model can include a Llama-2 model.

In at least some examples, the training of the one or more vision-language models 148 can include a number of stages. For example, a first stage of the training process of the one or more vision-language models 148 can include one or more operations to align the vision features of the image training data with corresponding text descriptions in the text feature space. The aligned data can then be provided to the one or more vision-language models 148. In various examples, a linear projection can be implemented to align the vision features and the text features. In one or more examples, the linear projection layer can be trained independently while other parameters of the one or more vision-language models 148 are frozen. The inputs during training can include image features and a description of the image features.

In one or more illustrative examples, a second stage of the training process for the one or more vision-language models 148 can include a continued training of the projection layer and adding in the use of LoRA techniques with respect to the weights of the one or more vision-language models 148. The objective of the training of the one or more vision-language models 148 can be a next token prediction. In one or more additional examples, cross-entropy loss can be used during the training process. In one or more additional illustrative examples, a third stage of the training process of the one or more vision-language models 148 can include implementing one or more reinforcement learning techniques. The one or more reinforcement learning techniques can be implemented to determine a truthfulness metric in relation to ground truth data. In various examples, one or more proximal policy optimization techniques can be applied with respect to the truthfulness metrics.

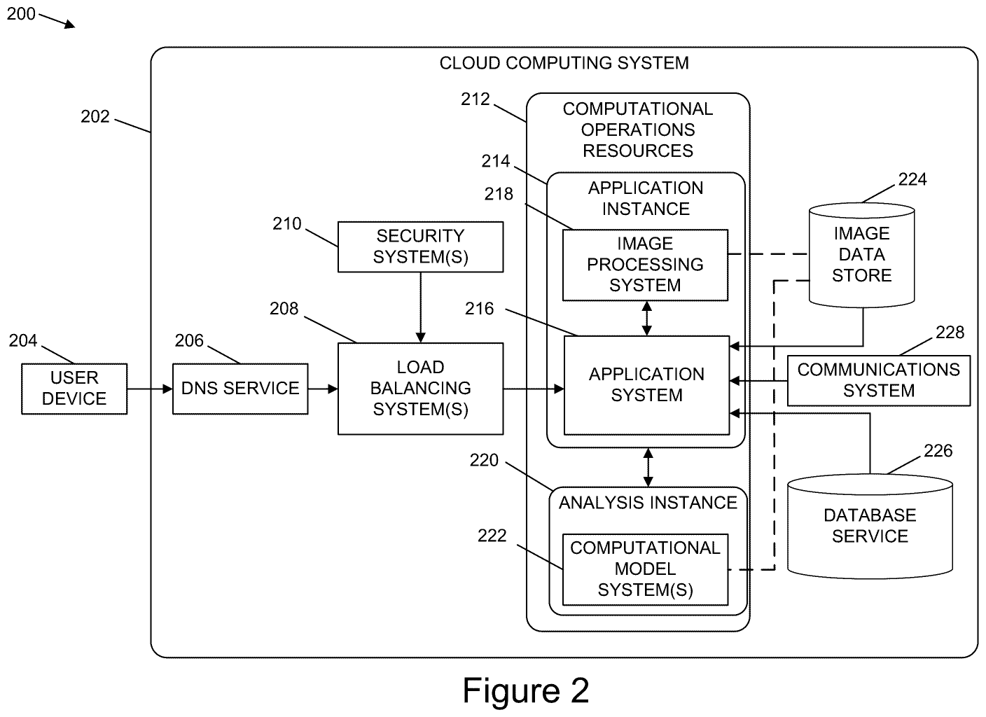

FIG. 2 is a diagram illustrating an example cloud computing architecture 200 to analyze biological imaging data, in accordance with one or more implementations. The cloud computing architecture 200 can include a cloud computing system 202 that implements one or more components and/or systems of the computing system 102 described with respect to FIG. 1. The cloud computing architecture 200 can also include a user device 204 that can access at least a portion of the functionality of the cloud computing system 202.

The cloud computing system 202 can include a DNS service 206. In response to receiving requests from the user device 204, the DNS service 206 can determine the resources of the cloud computing system 202 to access in response to the requests. Additionally, the cloud computing system 102 can include one or more load balancing systems 208. The one or more load balancing systems 208 can determine the resources of the cloud computing system 202 that have the capacity to handle various requests obtained from the user device 204 and direct execution of operations related to the requests to the resources with adequate capacity. Further, the cloud computing system 202 can include one or more security systems 212. The one or more security systems 212 can manage digital security certificates, such as secure sockets layer (SSL) certificates, for connecting users with the resources of the cloud computing system 202 that are used to implement one or more features of the computing system 102.

The cloud computing system 202 can also include one or more computational operations resources 212. The computational operations resources 212 can correspond to at least one of processing resources, memory resources, and/or other computational resources used to perform operations of the computing system 102. For example, the one or more computational operations resources 214 can include an application instance 214. The application instance 214 can correspond to operations performed with respect to at least one of a mobile application or a web-based application that enables access to functionality of the computing system 102. To illustrate, the application instance 214 can include an application system 216 the generates user interfaces and processes user input related to accessing functionality provided by the computing system 102 through the application. The application instance 214 also includes an image processing system 218 that provides access to images viewable via the application and that supports various operations that can be performed with respect to the images. The image processing system 218 can also identify different parts or layers of an image and provide access to particular portions of the images to be analyzed by the computational operations resources 212.

The one or more computational operations resources 214 can also include an analysis instance 220. The image analysis instance 220 can include one or more computational model systems 222. The one or more computational model systems 222 can perform operations to implement the one or more machine learning systems 142 described with respect to FIG. 1. For example, the one or more computational model systems 222 can implement one or more machine learning models to analyze images that are input to the computing system 102, determine whether or not a biological condition is present in subjects from which the images were obtained, and generate reports that include conclusions related to the images. In at least some examples, the computational model systems 222 can perform preprocessing with respect to image files such that the format of the image files is modified to a format that is supported by at least one of the application system 216 or the one or more computational model systems 222.

In one or more examples, the application system 220 can be in electronic communication with an image data store 224. The image data store 224 can store at least one of the training image data 114 or the subject image data 116 described with respect to FIG. 1. Further, the application system 220 can be in electronic communication with a database service 226. The database service 226 can provide a set of services to store and access data in a relational database, such as a MySQL database. In various examples, the application system 220 can be coupled to or otherwise access a communications system 228. The communications system 228 can include at least one of email or messaging functionality to enable communication to take place between at least one of users of the cloud computing system 202 or between the cloud computing system 202 and a user device 204.

In one or more illustrative examples, the application system 216 can perform registration and authentication operations and profile management operations to enable users to access the functionality provided by the computing system 102. The application system 216 can also provide access to the user device 204 to images stored in the image data store 224 as well as provide functionality to modify the images stored by the image data store 224. In at least some examples, the application system 216 can provide users with information indicating images that are able to be viewed and analyzed by the users and generate user interfaces that enable the users to view and modify the images. Additionally, the application system 216 can provide chat features that enable users to provide at least one of commands or questions that can be acted upon by the image processing system 218 and/or the analysis instance 220. In various examples, the application system 216 can at least one of request, access, or otherwise initiate services and/or computational resources of the cloud computing system 202 to perform operations described in relation to the computing system 102 described with respect to FIG. 1.

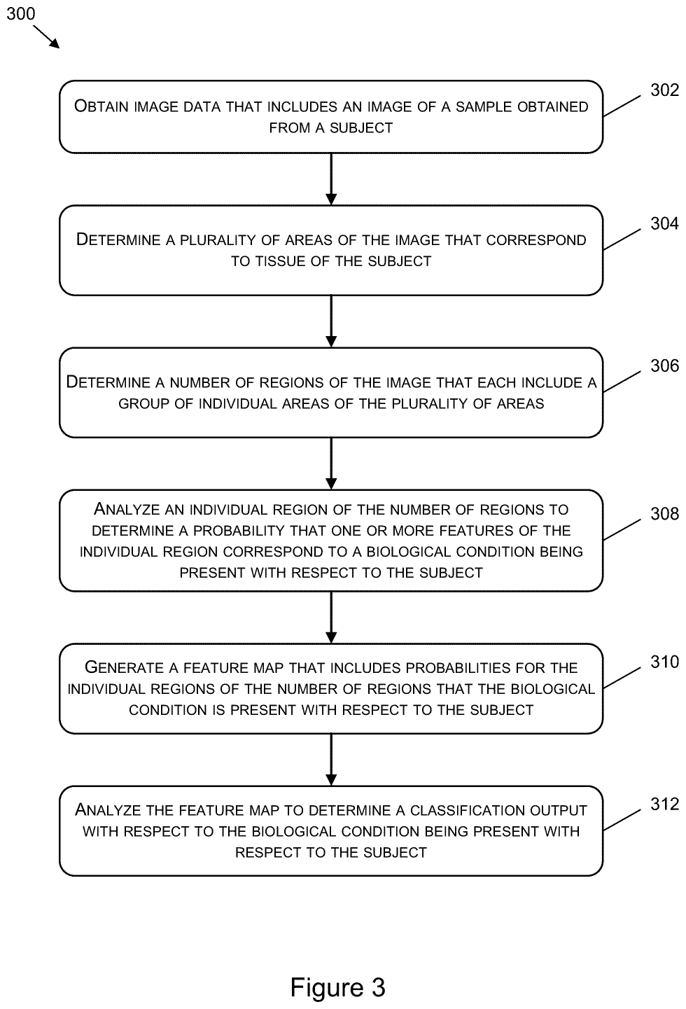

FIG. 3 is a flow diagram illustrating an example process 300 to analyze biological imaging data, in accordance with one or more implementations. The example processes are illustrated as collections of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

The process 300 can include, at 302, obtaining image data that includes an image of a sample obtained from a subject. In one or more examples, the image can include a biological image of a tissue sample obtained from the subject. In one or more additional examples, the image can correspond to a histology slide generated from the sample. In one or more illustrative examples, the image can include a whole slide image. In various examples, the image can be obtained using an application that analyzes images of tissue of subjects. The application can include a mobile device application or a web-based application. In at least some examples, the image can be displayed in a user interface of the application.

In at least some examples, after launching the application, at least one of text input or audio input can be analyzed to determine one or more operations to perform with respect to the image. In one or more illustrative examples, the at least one of text input or audio input can be captured via chat functionality of the application. The one or more operations to be performed with respect to the image can include at least one of applying a level of magnification to the image; rotating a view of the image; obtaining additional input that includes at least one of text annotations, video annotations, or audio annotations for the image; highlighting one or more portions of the image; or measuring distances between a number of attributes of the image. In one or more illustrative examples, the at least one of text input or audio input can include at least one of one or more commands or one or more questions directed to a request to analyze the image and determine the classification output related to the biological condition being present or being absent with respect to the subject. In various examples, the classification output can be provided in one or more user interfaces.

In addition, at 304, the process 300 can include determining a plurality of areas of the image that correspond to tissue of the subject. In various examples, the plurality of areas that correspond to tissue can be determined using one or more segmentation models. The one or more segmentation models can include a machine vision architecture that operates in conjunction with a transformer architecture. In one or more illustrative examples, the plurality of areas can correspond to patches of whole slide images that include tissue of the subject.

The transformer architectures can include a number of transformer blocks with individual transformer blocks having a number of neurons. In various examples, the transformer architecture can include at least 5 transformer blocks, at least 10 transformer blocks, at least 25 transformer blocks, at least 50 transformer blocks, at least 100 transformer blocks, at least 250 transformer blocks, at least 500 transformer blocks, or at least 1000 transformer blocks. In one or more examples, individual layers of neurons can include from 1 to 50,000 neurons, from 10 to 20,000 neurons, from 1000 to 10,000 neurons, from 1000 to 5000 neurons, from 5000 to 10,000 neurons, from 10,000 to 20,000 neurons, from 20,000 to 30,000 neurons, from 30,000 neurons to 40,000 neurons, or from 40,000 to 50,000 neurons. In at least some examples, the individual transformer blocks can include more than 50,000 neurons.

Additionally, individual neurons of the transformer architecture can implement an activation function. In one or more examples, neurons of the transformer architecture can implement a rectified linear unit activation function. In one or more additional examples, neurons of the transformer architecture can implement a Gaussian error linear unit activation function. In one or more further examples, neurons of the transformer architecture can implement a SoftMax activation function.

The training of the transformer architecture can be used to determine a number of weights of the transformer architecture. In one or more examples, the training process for the transformer architecture can determine weights of connections between neurons included in transformer blocks of the transformer machine learning architecture. Initially, the weights can be randomly assigned. In one or more additional examples, the training of the transformer architecture can be performed over at least 500 iterations, at least 1000 iterations, at least 2500 iterations, at least 5000 iterations, at least 10,000 iterations, at least 25,000 iterations, or at least 50,000 iterations. The training can be performed over at least 5 epochs, at least 10 epochs, at least 15 epochs, at least 20 epochs, at least 25 epochs, at least 30 epochs, at least 40 epochs, at least 50 epochs, at least 60 epochs, at least 70 epochs, at least 80 epochs, at least 90 epochs, or at least 100 epochs.

The process 300 can also include, at 306, determining a number of regions of the image that each include a group of individual areas of the plurality of areas. The regions can be generated by combining individual areas of the plurality of areas. In at least some examples, a tissue mask can be generated by assembling patches of whole slide images that correspond to tissue of the subject.

Further, at 308, the process 300 can include analyzing an individual region of the number of regions to determine a probability that one or more features of the individual region correspond to a biological condition being present with respect to the subject. At 310, the process 300 can include generating a feature map that includes probabilities for the individual regions of the number of regions that the biological condition is present with respect to the subject. In one or more illustrative examples, individual probabilities can correspond to individual pixels included in the image and the individual probabilities for the individual pixels can be combined to produce the feature map.

Additionally, the process 300 can include, at 312, analyzing the feature map to determine a classification output with respect to the biological condition being present with respect to the subject. In at least some instances, the classification output can indicate that the subject from which the tissue sample was extracted has a biological condition. In other scenarios, the classification output can indicate that a biological condition is absent with respect to the subject. In one or more examples, feature map and the classification output can be analyzed using a large vision language model to generate a report. The report can include information about the image and can include conclusions about the image, such as the classification output. Further, at least a portion of the report can be viewed in an additional user interface of the application. In one or more illustrative examples, the large visional language model can generate a mapping of features of the image with text data to generate the report. In various examples, the large vision training model can be trained on a corpus of data that includes training images and that includes, for individual training images, text information corresponding to features of the individual training images.

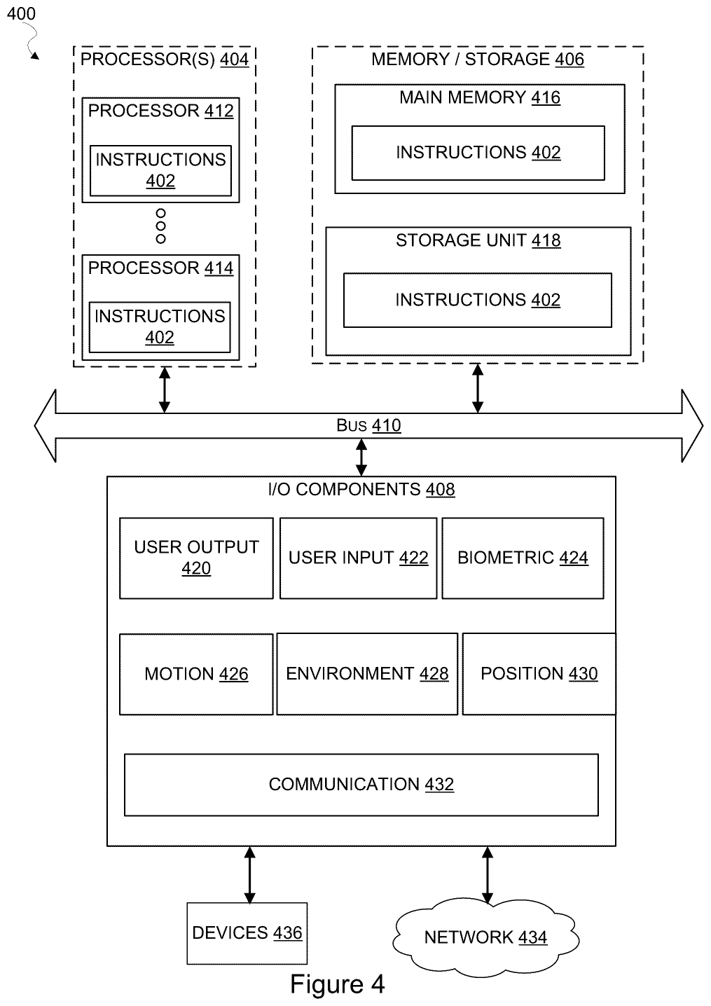

FIG. 4 is a block diagram illustrating components of a machine 400, according to some example implementations, able to read instructions from a machine-readable medium (e.g., a machine-readable storage medium) and perform any one or more of the methodologies discussed herein. Specifically, FIG. 4 shows a diagrammatic representation of the machine 400 in the example form of a computer system, within which instructions 402 (e.g., software, a program, an application, an applet, an app, or other executable code) for causing the machine 400 to perform any one or more of the methodologies discussed herein may be executed. As such, the instructions 402 may be used to implement modules or components described herein. The instructions 402 transform the general, non-programmed machine 400 into a particular machine 400 programmed to carry out the described and illustrated functions in the manner described. In alternative implementations, the machine 400 operates as a standalone device or may be coupled (e.g., networked) to other machines. In a networked deployment, the machine 400 may operate in the capacity of a server machine or a client machine in a server-client network environment, or as a peer machine in a peer-to-peer (or distributed) network environment. The machine 400 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 402, sequentially or otherwise, that specify actions to be taken by machine 400. Further, while only a single machine 400 is illustrated, the term “machine” shall also be taken to include a collection of machines that individually or jointly execute the instructions 402 to perform any one or more of the methodologies discussed herein.

The machine 400 may include processors 404, memory/storage 406, and I/O components 408, which may be configured to communicate with each other such as via a bus 410. In an example implementation, the processors 404 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 412 and a processor 414 that may execute the instructions 402. The term “processor” is intended to include multi-core processors 404 that may comprise two or more independent processors (sometimes referred to as “cores”) that may execute instructions 402 contemporaneously. Although FIG. 4 shows multiple processors 404, the machine 400 may include a single processor 412 with a single core, a single processor 412 with multiple cores (e.g., a multi-core processor), multiple processors 412, 414 with a single core, multiple processors 412, 414 with multiple cores, or any combination thereof.

The memory/storage 406 may include memory, such as a main memory 416, or other memory storage, and a storage unit 418, both accessible to the processors 404 such as via the bus 410. The storage unit 418 and main memory 416 store the instructions 402 embodying any one or more of the methodologies or functions described herein. The instructions 402 may also reside, completely or partially, within the main memory 416, within the storage unit 418, within at least one of the processors 404 (e.g., within the processor's cache memory), or any suitable combination thereof, during execution thereof by the machine 400. Accordingly, the main memory 416, the storage unit 418, and the memory of processors 404 are examples of machine-readable media.

The I/O components 408 may include a wide variety of components to receive input, provide output, produce output, transmit information, exchange information, capture measurements, and so on. The specific I/O components 408 that are included in a particular machine 400 will depend on the type of machine. For example, portable machines such as mobile phones will likely include a touch input device or other such input mechanisms, while a headless server machine will likely not include such a touch input device. It will be appreciated that the I/O components 408 may include many other components that are not shown in FIG. 4. The I/O components 408 are grouped according to functionality merely for simplifying the following discussion and the grouping is in no way limiting. In various example implementations, the I/O components 408 may include user output components 420 and user input components 422. The user output components 420 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 422 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 408 may include biometric components 424, motion components 426, environmental components 428, or position components 430 among a wide array of other components. For example, the biometric components 424 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 426 may include acceleration sensor components (e.g., accelerometer), gravitation sensor components, rotation sensor components (e.g., gyroscope), and so forth. The environmental components 428 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 430 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 408 may include communication components 432 operable to couple the machine 400 to a network 434 or devices 436. For example, the communication components 432 may include a network interface component or other suitable device to interface with the network 434. In further examples, communication components 432 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 436 may be another machine 400 or any of a wide variety of peripheral devices (e.g., a peripheral device coupled via a USB).

Moreover, the communication components 432 may detect identifiers or include components operable to detect identifiers. For example, the communication components 432 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 432, such as location via Internet Protocol (IP) geo-location, location via Wi-Fi® signal triangulation, location via detecting an NFC beacon signal that may indicate a particular location, and so forth.

As used herein, “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations. For example, a hardware component may include software executed by a general-purpose processor 404 or other programmable processor. Once configured by such software, hardware components become specific machines (or specific components of a machine 400) uniquely tailored to perform the configured functions and are no longer general-purpose processors 404. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering implementations in which hardware components are temporarily configured (e.g., programmed), each of the hardware components need not be configured or instantiated at any one instance in time. For example, where a hardware component comprises a general-purpose processor 404 configured by software to become a special-purpose processor, the general-purpose processor 404 may be configured as respectively different special-purpose processors (e.g., comprising different hardware components) at different times. Software accordingly configures a particular processor 412, 414 or processors 404, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices, and can operate on a resource (e.g., a collection of information). The various operations of example methods described herein may be performed, at least partially, by one or more processors 404 that are temporarily configured (e.g., by software) or permanently configured to perform the relevant operations. Whether temporarily or permanently configured, such processors 404 may constitute processor-implemented components that operate to perform one or more operations or functions described herein. As used herein, “processor-implemented component” refers to a hardware component implemented using one or more processors 404. Similarly, the methods described herein may be at least partially processor-implemented, with a particular processor 412, 414 or processors 404 being an example of hardware. For example, at least some of the operations of a method may be performed by one or more processors 404 or processor-implemented components. Moreover, the one or more processors 404 may also operate to support performance of the relevant operations in a “cloud computing” environment or as a “software as a service” (SaaS). For example, at least some of the operations may be performed by a group of computers (as examples of machines 400 including processors 404), with these operations being accessible via a network 434 (e.g., the Internet) and via one or more appropriate interfaces (e.g., an API). The performance of certain of the operations may be distributed among the processors, not only residing within a single machine 400, but deployed across a number of machines. In some example implementations, the processors 404 or processor-implemented components may be located in a single geographic location (e.g., within a home environment, an office environment, or a server farm). In other example implementations, the processors 404 or processor-implemented components may be distributed across a number of geographic locations.

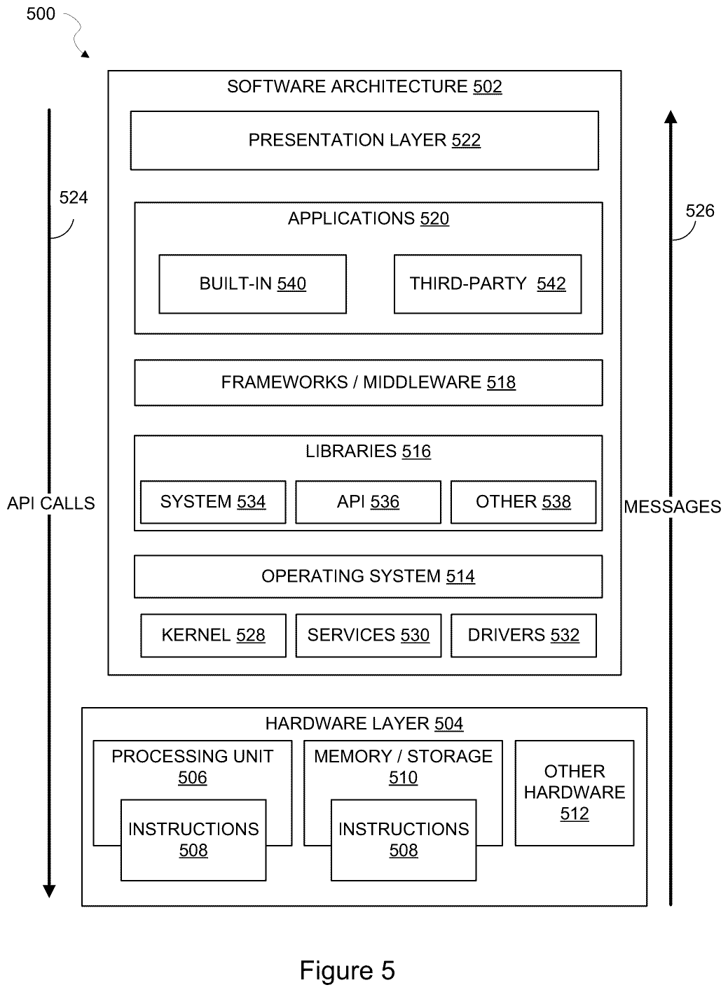

FIG. 5 is a block diagram illustrating system 500 that includes an example software architecture 502, which may be used in conjunction with various hardware architectures herein described. FIG. 5 is a non-limiting example of a software architecture, and it will be appreciated that many other architectures may be implemented to facilitate the functionality described herein. The software architecture 502 may execute on hardware such as machine 400 of FIG. 4 that includes, among other things, processors 404, memory/storage 406, and input/output (I/O) components 408. A representative hardware layer 504 is illustrated and can represent, for example, the machine 400 of FIG. 4. The representative hardware layer 504 includes a processing unit 506 having associated executable instructions 508. Executable instructions 508 represent the executable instructions of the software architecture 502, including implementation of the methods, components, and so forth described herein. The hardware layer 504 also includes at least one of memory or storage modules memory/storage 510, which also have executable instructions 508. The hardware layer 504 may also comprise other hardware 512.

In the example architecture of FIG. 5, the software architecture 502 may be conceptualized as a stack of layers where each layer provides particular functionality. For example, the software architecture 502 may include layers such as an operating system 514, libraries 516, frameworks/middleware 5518, applications 520, and a presentation layer 522. Operationally, the applications 520 or other components within the layers may invoke API calls 524 through the software stack and receive messages 526 in response to the API calls 524. The layers illustrated are representative in nature and not all software architectures have all layers. For example, some mobile or special purpose operating systems may not provide a frameworks/middleware 518, while others may provide such a layer. Other software architectures may include additional or different layers.

The operating system 514 may manage hardware resources and provide common services. The operating system 514 may include, for example, a kernel 528, services 530, and drivers 532. The kernel 528 may act as an abstraction layer between the hardware and the other software layers. For example, the kernel 528 may be responsible for memory management, processor management (e.g., scheduling), component management, networking, security settings, and so on. The services 530 may provide other common services for the other software layers. The drivers 532 are responsible for controlling or interfacing with the underlying hardware. For instance, the drivers 532 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 516 provide a common infrastructure that is used by at least one of the applications 520, other components, or layers. The libraries 516 provide functionality that allows other software components to perform tasks in an easier fashion than to interface directly with the underlying operating system 514 functionality (e.g., kernel 528, services 530, drivers 532). The libraries 516 may include system libraries 534 (e.g., C standard library) that may provide functions such as memory allocation functions, string manipulation functions, mathematical functions, and the like. In addition, the libraries 516 may include API libraries 536 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 516 may also include a wide variety of other libraries 538 to provide many other APIs to the applications 520 and other software components/modules.

The frameworks/middleware 518 (also sometimes referred to as middleware) provide a higher-level common infrastructure that may be used by the applications 520 or other software components/modules. For example, the frameworks/middleware 518 may provide various graphical user interface functions, high-level resource management, high-level location services, and so forth. The frameworks/middleware 518 may provide a broad spectrum of other APIs that may be utilized by the applications 520 or other software components/modules, some of which may be specific to a particular operating system 514 or platform.

The applications 520 include built-in applications 1040 and third-party applications 1042. Examples of representative built-in applications 1040 may include, but are not limited to, a contacts application, a browser application, a book reader application, a location application, a media application, a messaging application, or a game application. Third-party applications 1042 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 1042 may invoke the API calls 524 provided by the mobile operating system (such as operating system 514) to facilitate functionality described herein.

The applications 520 may use built-in operating system functions (e.g., kernel 528, services 530, drivers 532), libraries 516, and frameworks/middleware 518 to create UIs to interact with users of the system. Alternatively, or additionally, in some systems, interactions with a user may occur through a presentation layer, such as presentation layer 522. In these systems, the application/component “logic” can be separated from the aspects of the application/component that interact with a user.

At least some of the processes described herein can be embodied in computer-readable instructions for execution by one or more processors such that the operations of the processes may be performed in part or in whole by the functional components of one or more computer systems. Accordingly, computer-implemented processes described herein are by way of example with reference thereto, in some situations. However, in other implementations, at least some of the operations of the computer-implemented processes described herein can be deployed on various other hardware configurations. The computer-implemented processes described herein are therefore not intended to be limited to the systems and configurations described with respect to FIGS. 4 and 5 and can be implemented in whole, or in part, by one or more additional system and/or components.

Although the flowcharts described herein can show operations as a sequential process, many of the operations can be performed in parallel or concurrently. In addition, the order of the operations can be re-arranged. A process is terminated when its operations are completed. A process can correspond to a method, a procedure, an algorithm, etc. The operations of methods may be performed in whole or in part, can be performed in conjunction with some or all of the operations in other methods, and can be performed by any number of different systems, such as the systems described herein, or any portion thereof, such as a processor included in any of the systems.

### EXAMPLES

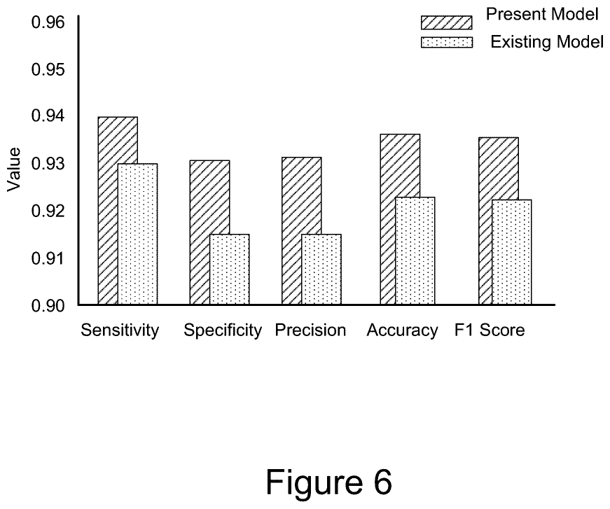

FIG. 6 illustrates a comparison of a number of classification metrics for an example architecture described herein that analyzes biological imaging data with respect to an existing architecture that analyzes biological imaging data.

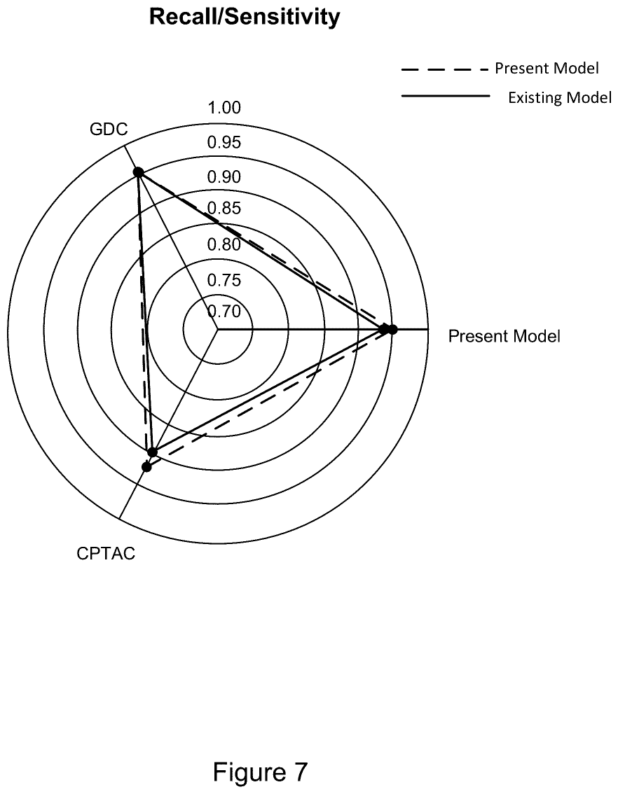

FIG. 7 illustrates a comparison of recall/sensitivity for an example architecture described herein that analyzes biological imaging data with respect to an existing architecture that analyzes biological imaging data.

What is claimed is:

1. A method comprising:
obtaining, by a computing system including one or more computing devices having one or more processors and memory, image data that includes an image of a sample obtained from a subject;
determining, by the computing system, a plurality of areas of the image that correspond to tissue of the subject;
determining, by the computing system, a number of regions of the image that each include a group of individual areas of the plurality of areas;
analyzing, by the computing system, an individual region of the number of regions to determine a probability that one or more features of the individual region correspond to a biological condition being present with respect to the subject;
generating, by the computing system, a feature map that includes probabilities for the individual regions of the number of regions that the biological condition is present with respect to the subject; and
analyzing, by the computing system, the feature map to determine a classification output related to the biological condition being present with respect to the subject.

2. The method of claim 1, wherein the image corresponds to at least one histology slide generated from the sample obtained from the subject.

3. The method of claim 1, wherein:
the individual region of the number of regions is analyzed using a segmentation model that is implemented with respect to each region of the number of regions;
an output of the segmentation model includes individual probabilities for individual pixels included in the number of regions indicating that the biological condition is present with respect to the subject; and
the individual probabilities for the individual pixels are combined to produce the feature map.

4. The method of claim 3, wherein the segmentation model includes a machine vision architecture in conjunction with a first transformer-based architecture and the classification output is generated by implementing a classification model that includes a second transformer-based architecture.

5. The method of claim 4, wherein at least one of at least a portion of the segmentation model or the classification model are implemented using one or more application specific integrated circuits (ASICs).

6. The method of claim 1, comprising:
analyzing, by the computing system, the feature map and the classification output using a large-vision-language model to generate a report that is displayed by one or more display devices and that includes information about the image and that includes the classification output for the sample.

7. The method of claim 6, wherein:
the large-vision-language model generates a mapping of features of the image with text data to generate the report; and
the large-vision-language model is trained on a corpus of data that, for a number of individual training images, indicates text information about features of the individual training images.

8. The method of claim 1, comprising:
receiving, by the computing system, a request to launch an application that analyzes images of tissue of subjects;
obtaining, by the computing system and within the application, at least one of text input or audio input via one or more input devices of a computing device; and
analyzing, by the computing system, the at least one of the text input or the audio input to determine one or more operations to perform with respect to the image.

9. The method of claim 8, wherein the one or more operations include:
applying a level of magnification to the image;
rotating a view of the image;
obtaining additional input that includes at least one of text annotations, video annotations, or audio annotations for the image;
highlighting one or more portions of the image; or
measuring distances between a number of attributes of the image.

10. The method of claim 8, wherein the at least one of the text input or the audio input includes a request to analyze the image and determine the classification output related to the biological condition being present with respect to the subject.

11. A system comprising:
one or more hardware processors; and
memory storing computer-readable instructions that, when executed by the one or more hardware processors cause the one or more hardware processors to perform operations comprising:
obtaining image data that includes an image of a sample obtained from a subject;
determining a plurality of areas of the image that correspond to tissue of the subject;
determining a number of regions of the image that each include a group of individual areas of the plurality of areas;
analyzing an individual region of the number of regions to determine a probability that one or more features of the individual region correspond to a biological condition being present with respect to the subject;
generating a feature map that includes probabilities for the individual regions of the number of regions that the biological condition is present with respect to the subject; and
analyzing the feature map to determine a classification output related to the biological condition being present with respect to the subject.

12. The system of claim 11, wherein the image corresponds to at least one histology slide generated from the sample obtained from the subject and the biological condition corresponds to at least one of a skin cancer or a colorectal cancer.

13. The system of claim 11, wherein the memory stores additional computer-readable instructions that, when executed by the one or more hardware processors cause the one or more hardware processors to perform additional operations comprising:
performing a training process for a segmentation model using curated training data that includes a number of training images that have been annotated, wherein the training process is performed to achieve a threshold value for a combination of focal loss and dice loss for the segmentation model and the number of training images correspond a plurality of initial images of tissue of training subjects such that the number of training images have a greater level of magnification and a lower level of resolution than the plurality of initial images.

14. The system of claim 11, wherein the memory stores additional computer-readable instructions that, when executed by the one or more hardware processors cause the one or more hardware processors to perform additional operations comprising:
performing an additional training process for a classification model that implements a transformer-based architecture, wherein the additional training process is performed with respect to threshold value of cross-entropy loss and with label smoothing.

15. The system of claim 14, wherein:
the additional training process includes, for each epoch of the additional training process, determining validation metrics that include at least one of accuracy, precision, recall, specificity, or Jaccard Index; and
the additional training process continues until values for the validation metrics are optimized.

16. The system of claim 11, wherein the memory stores additional computer-readable instructions that, when executed by the one or more hardware processors cause the one or more hardware processors to perform additional operations comprising:
generating one or more user interfaces that include the classification output.

17. The system of claim 16, wherein the one or more user interfaces include user interface elements that are selectable to perform at least one of applying a level of magnification to one or more images; rotating a view of the one or more images; obtaining additional input that includes at least one of text annotations, video annotations, or audio annotations for the one or more images; highlighting one or more portions of the one or more images; or measuring distances between a number of attributes of the one or more images.

18. One or more non-transitory computer-readable storage media including computer-readable instructions that, when executed by one or more processing devices, perform operations comprising;
obtaining image data that includes an image of a sample obtained from a subject;
determining a plurality of areas of the image that correspond to tissue of the subject;
determining a number of regions of the image that each include a group of individual areas of the plurality of areas;
analyzing an individual region of the number of regions to determine a probability that one or more features of the individual region correspond to a biological condition being present with respect to the subject;
generating a feature map that includes probabilities for the individual regions of the number of regions that the biological condition is present with respect to the subject; and
analyzing the feature map to determine a classification output related to the biological condition being present with respect to the subject.

19. The one or more non-transitory computer-readable storage media of claim 18, wherein:
the individual region of the number of regions is analyzed using a segmentation model that is implemented with respect to each region of the number of regions, the segmentation model including a machine vision architecture in conjunction with a first transformer-based architecture;
an output of the segmentation model includes individual probabilities for individual pixels included in the number of regions indicating that the biological condition is present with respect to the subject;
the individual probabilities for the individual pixels are combined to produce the feature map; and
the classification output is generated by implementing a classification model that includes a second transformer-based architecture.

20. The one or more non-transitory computer-readable storage media of claim 19, comprising additional computer-readable instructions that, when executed by the one or more processing devices, perform additional operations comprising:
performing a training process for the segmentation model using curated training data that includes a number of training images that have been annotated, wherein the training process is performed to achieve a threshold value for a combination of focal loss and dice loss for the segmentation model and the number of training images correspond a plurality of initial images of tissue of training subjects such that the number of training images have a greater level of magnification and a lower level of resolution than the plurality of initial images.
